(19) United States
(12) Patent Application Publication
(10) Pub. No.: US 2026/0224277 A1
(43) Pub. Date: Aug. 6, 2026

(54) SURGICAL SYSTEM AND METHOD FOR GENERATING SENSOR DATA DURING THERAPEUTIC ENERGY DELIVERY

(71) Applicant: Cilag GmbH International, Zug (CH)

(72) Inventor: Joseph P. Murphy, Cincinnati, OH (US)

(21) Appl. No.: 19/043,865

(22) Filed: Feb. 3, 2025

(51) Int. Cl. A61B 18/14; A61B 17/00; A61B 18/00; A61B 90/00

(52) U.S. Cl. CPC A61B 18/1445; A61B 2017/00221; A61B 2018/00607; A61B 2018/0063; A61B 2018/00702; A61B 2090/061

(57) ABSTRACT

A surgical system includes an end effector and a processor. The end effector includes a plurality of electrodes configured to contact a tissue of a patient and a first sensor configured to generate a first measurement during use. The processor is configured to control delivery of a therapeutic radio frequency (RF) energy signal based on an energy delivery algorithm to the plurality of electrodes and initiate a sensing window of time defined by pausing the delivery of the therapeutic RF energy signal for a duration of time whereafter the delivery of the therapeutic RF energy signal resumes. Additionally, the processor is configured to, during the sensing window, generate a first measurement from the first sensor and transmit end effector data, which comprises the first measurement from the first sensor, to the surgical system.

### BACKGROUND

A variety of surgical systems include a surgical instrument with one or more elements that transmit radio frequency (RF) energy to tissue (e.g., to coagulate or seal the tissue). An example of such an electrosurgical instrument is the ENSEAL® Tissue Sealing Device by Ethicon Endo-Surgery, Inc., of Cincinnati, Ohio. Contemporary electrosurgical instruments use sensors located distally at an end effector of the instrument to make various measurements that can be related to the instrument or the environment, such as a precise jaw aperture measurement when the end effector includes first and second jaws. These measurements are useful when made during the transmission of therapeutic RF energy. However, measurements made during the transmission of therapeutic RF energy are subject to a large amount of electrical noise, which can distort and obscure the measurements. The use of filtering circuits or electrical shielding can help reduce electrical noise but introduce additional complexity and cost to the design of an electrosurgical instrument.

While a variety of surgical systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts an exploded perspective view of the end effector of FIG. 2;

FIG. 5 depicts a flowchart illustrating one method for generating sensor data during delivery of a therapeutic radio frequency (RF) energy signal in a sensing window with the electrosurgical instrument of FIG. 1;

FIG. 6 depicts a graph which includes an illustration of an oscilloscope capture of multiple sensing windows during delivery of a therapeutic RF energy signal with the electrosurgical instrument of FIG. 1 when the method of FIG. 5 is applied; and

FIGS. 7A and 7B depict graphs which include illustrative examples of the jaw aperture measured over time by the electrosurgical instrument of FIG. 1 when the method of FIG. 5 is not applied (7A) and when the method of FIG. 5 is applied (7B).

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

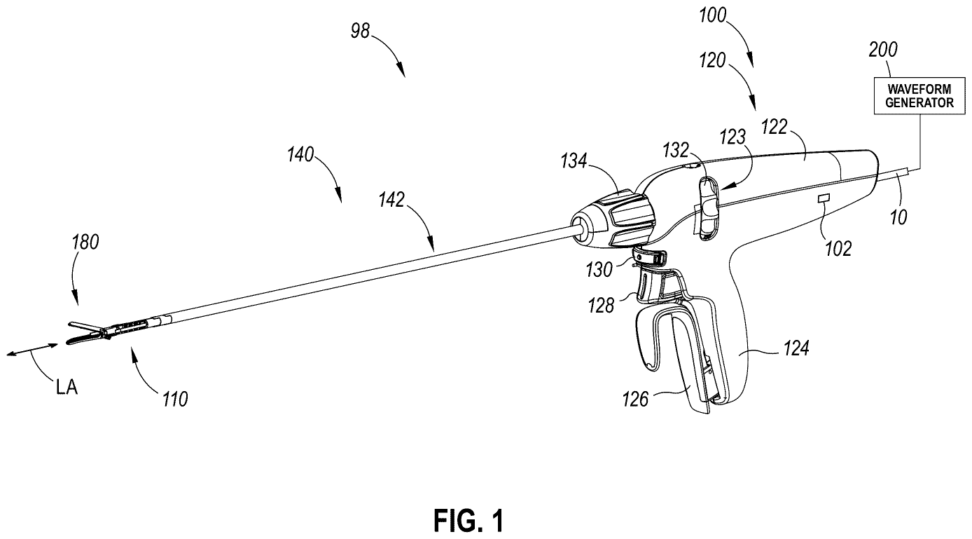

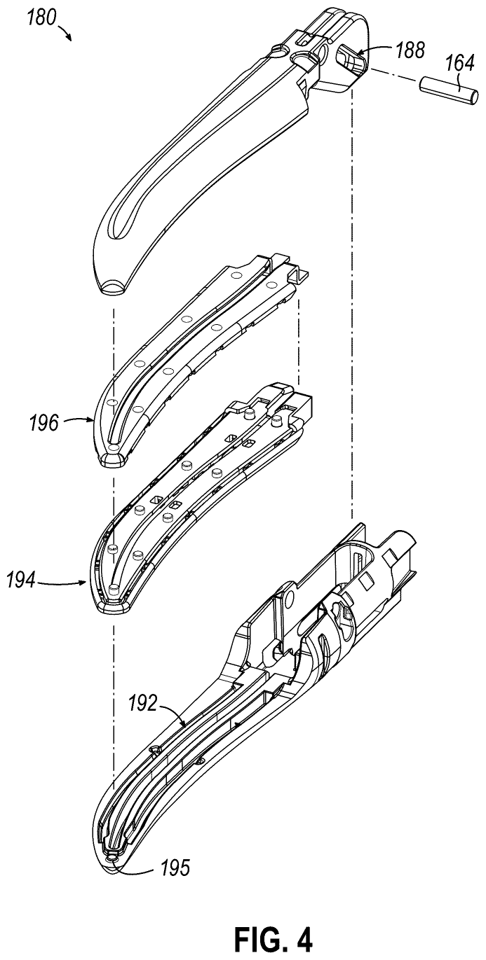

FIGS. 1-4 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to apply a non-therapeutic bipolar radio frequency (RF) energy in order to identify and/or verify that the correct tissue is present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a waveform generator (200) of surgical system (98), which is capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Although electrosurgical instrument (100) is primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). While in the present example control unit (102) is included in handle assembly (120), other embodiments may include control unit (102), including possible additional control units (not shown) that perform one or more functions of control unit (102), at another location of the electrosurgical instruments (100), or another component of surgical system (98). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) will bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

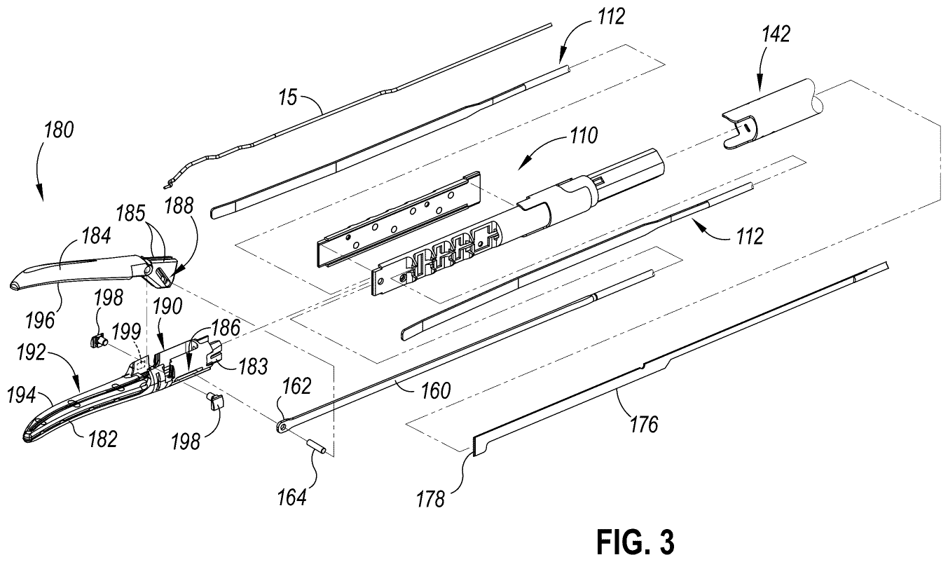

Shaft assembly (140) includes distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between distal a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrodes (194, 196).

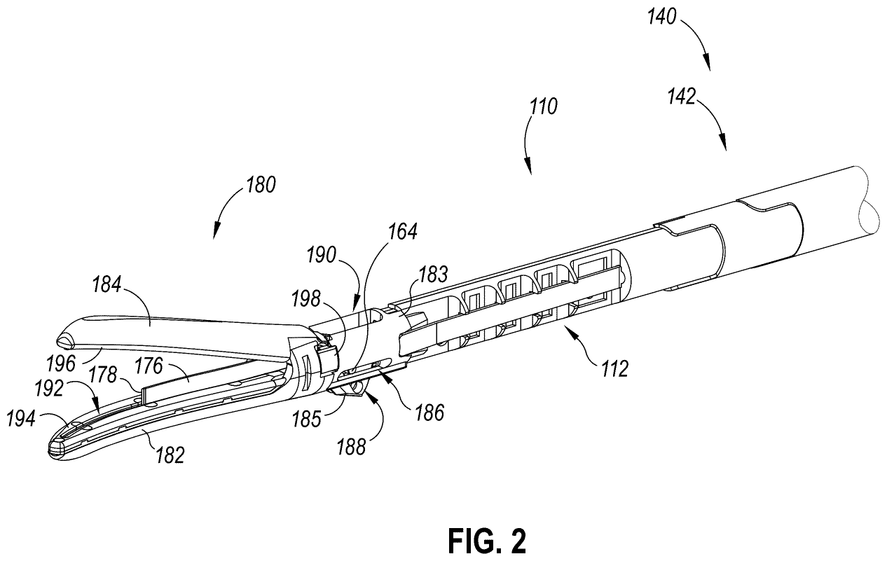

As best seen in FIGS. 2-4, end effector (180) includes lower jaw (182) pivotably coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

Lower jaw (182) also includes a jaw angle sensor (199) to measure the relative jaw angle between lower jaw (182) and upper jaw (184) during energy delivery. The jaw angle sensor (199) may be configured similar to the force sensor (195) to communicate its measurements to control unit (102). While in the current example, force sensor (195) and jaw angle sensor (199) are incorporated into electrosurgical instrument (100) and are in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor communicates signals to control unit (102) indicative of the degree end effector 180 is deflected from the longitudinal axis (LA) by articulation control (132) and articulation assembly (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise a respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that is slidably coupled along proximal portion of shaft assembly (140). Knife coupling body is coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body is coupled to knife member (176), knife member (176) translates distally within shaft assembly (140), articulation assembly (110), and within knife pathway (192) of end effector (180). Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) is retracted. Next, the operator may release jaw closure trigger (126) such that jaws (182, 184) pivot into the opened configuration, releasing tissue.

### II. Sensor Measurement Window

In an attempt to obtain end effector data from sensors of an end effector during the delivery of a therapeutic radio frequency (RF) energy signal driven by an energy delivery algorithm without the distortion caused by electrical noise and without the added complexity and cost of filtering circuits and electrical shielding, additional methods have been explored. One such approach introduces a brief pause—a sensor measurement window, or sensing window—during the delivery of the therapeutic RF energy signal. Following this approach, the end effector data can be obtained during the pause without any distortion. In other words, and using the surgical system (98) as an example, control unit (102) may query one or more sensors of end effector (180), generate measurement results from the query, and transmit the results to another component of the surgical system (98) for further processing, such as a computer (not shown), without the electrical noise caused by the therapeutic RF energy signal. Because the pause is brief, e.g., 10 ms, the impact to the energy delivery algorithm is insignificant. While surgical system (98) is being used to demonstrate the approach, it should be appreciated that the method described herein may also be applied to other surgical systems, including those that use advanced bipolar (ABP), harmonic, monopolar, and bipolar devices. The other surgical systems may also include different sensors placed distally on an electrosurgical instrument, such as sensors related to (1) tissue sensing and (2) stapler use (e.g., type of reload, state of the reload[fired/authentic]).

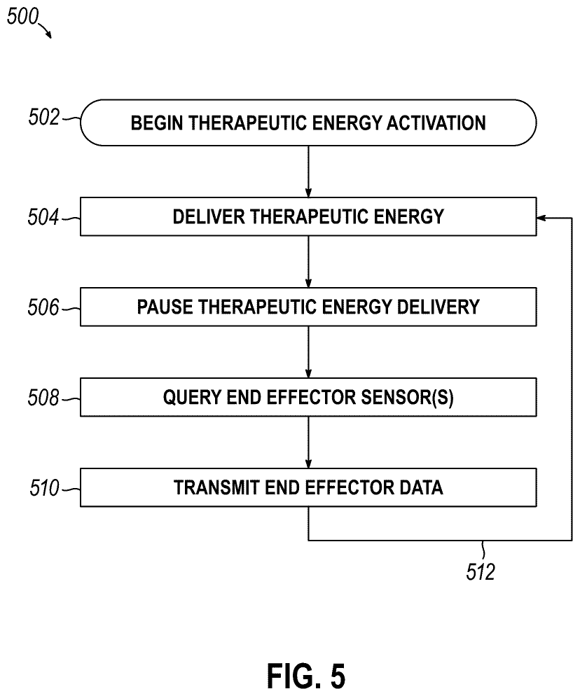

FIG. 5 depicts a flowchart illustrating one method, method (500), for generating sensor data during delivery of a therapeutic radio frequency (RF) energy signal in a sensing window with the electrosurgical instrument (100) of FIG. 1. Initially, a therapeutic energy delivery algorithm is activated (502). Activation may occur by way of user selection on the electrosurgical instrument (100) or another component of the surgical system (98), automatically based on the operation of end effector (180), or another method apparent to those skilled in the art. The therapeutic energy delivery algorithm defines how therapeutic RF energy is to be delivered to the electrodes (194, 196) of electrosurgical equipment (100). As one example, when the therapeutic energy delivery algorithm is a harmonic algorithm such as a harmonic advanced hemostasis algorithm, the algorithm may comprise one or more sealing phases and one or more cutting phases, each of which alters the RF energy being delivered. Other non-limiting examples of therapeutic energy delivery algorithms include advanced bipolar (ABP), monopolar, and bipolar.

Therapeutic energy is then delivered (504) to the electrodes (194, 196) of electrosurgical instrument (100) according to the activated therapeutic energy delivery algorithm. A sensing window is introduced to pause (506) delivery of therapeutic energy. The sensing window may not adversely impact the cutting performed during the cutting phase. The pause may last the amount of time needed to obtain end effector data, such as any duration up to 10 ms. In some embodiments, the pause is imperceptible to a user of the electrosurgical instrument (100). In other words, a user operating electrosurgical instrument (100) would not observe a deviation from the activated therapeutic energy delivery algorithm. For example, if a user is operating electrosurgical instrument (100) during a cutting phase of a therapeutic energy delivery algorithm and the sensing window is introduced during the cutting phase, the user never ceases to think the electrosurgical instrument (100) is cutting.

During the sensing window, one or more sensors from the end effector (180), such as jaw angle sensor (199), may be queried (508) one or more times. Here, and using jaw angle sensor (199) as an example, one or more measurements of the jaw aperture based on a position of the lower jaw (182) relative to the upper jaw (184) may be generated from the jaw angle sensor (199). In some embodiments, the one or more measurements are then transmitted (510) as part of end effector data from the electrosurgical instrument (100) to another component of the surgical system (98) during the sensing window. The end effector data may comprise various types of data, such as sensor data, that originates or is derived from end effector (180). Transmission of end effector data may occur via a direct wired connection from the electrosurgical instrument (100), wireless communication methods that leverage near-field communication (NFC) or Bluetooth^{®}connections, or any another communication method known to those skilled in the art. By generating and transmitting during the sensing window, the distortion caused by electrical noise stemming from the therapeutic RF energy signal is avoided without any additional complexity or components.

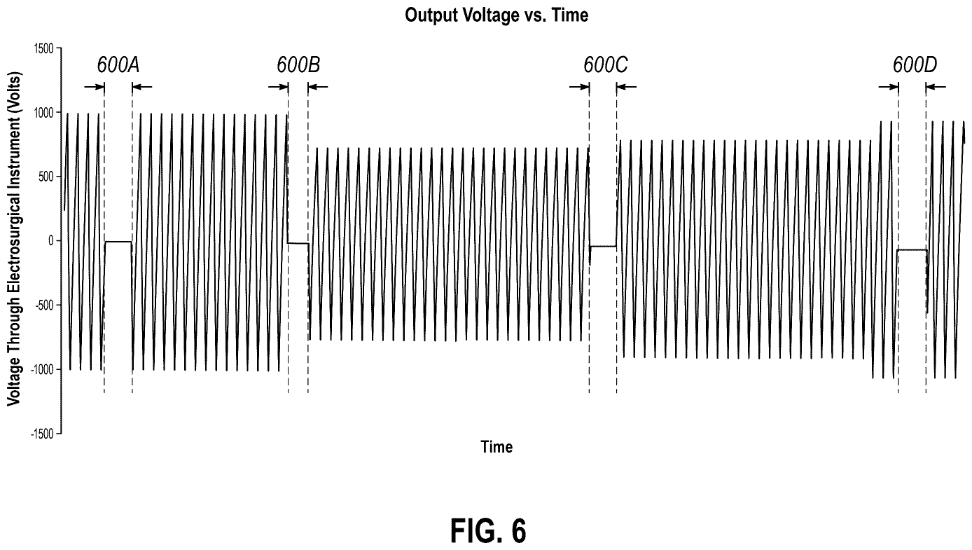

As illustrated in FIG. 5 by the loop (512), multiple sensing windows may occur during the activated therapeutic energy delivery algorithm. In one embodiment, the sensing windows may be periodic such that multiple sensing windows occur at a regular interval of time. Another embodiment may introduce sensing windows sporadically such that there is no regular interval of time in between sensing windows, as shown by sporadic sensing windows (600A, 600B, 600C, 600D) of FIG. 6. In yet another embodiment, a sensing window may be introduced at one or more key areas of interest of the therapeutic energy delivery algorithm. For example, a sensing window may be introduced between the sealing and cutting phases of a harmonic advanced hemostasis algorithm.

Additionally, in some embodiments, the size of the sensing window (i.e., the duration of the pause of the therapeutic RF energy signal) may be dynamic such that it varies from sensing window to sensing window. This may be the case, as one example, where the end effector data has been transmitted before the defined pause of a sensing window (e.g., at 4 ms of a 10 ms pause) is completed. Here, the sensing window would conclude before the end of the defined pause (e.g., 10 ms) to restore delivery of the therapeutic RF energy signal as soon as possible. As another example, the reaching of a threshold (e.g., a target jaw aperture) could trigger the conclusion or extension of the sensing window.

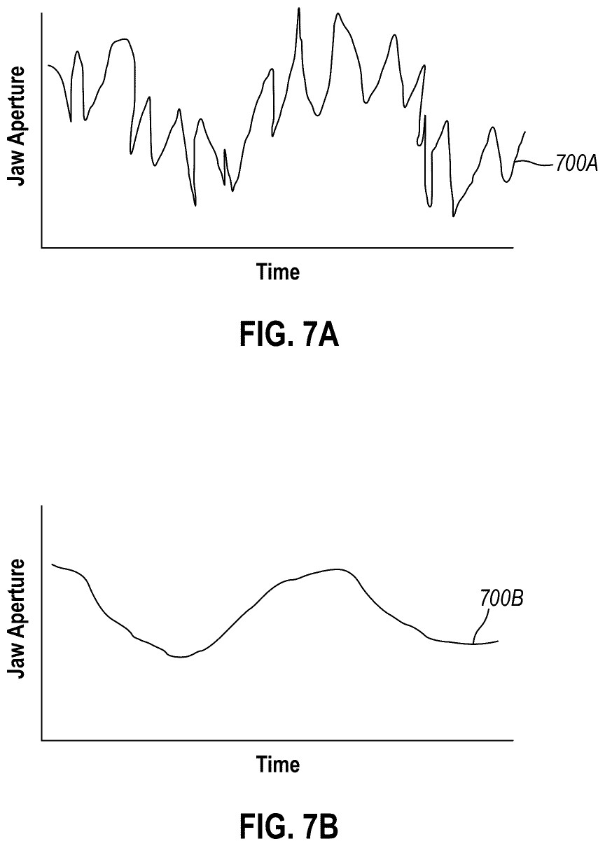

FIGS. 7A and 7B depict graphs which include illustrative examples of jaw aperture measurements (700A, 700B) generated over time from the jaw angle sensor (199) of the electrosurgical instrument (100). Jaw aperture measurement (700A) of FIG. 7A illustrates the distortion produced while generating the measurements from the jaw angle sensor (199) during the delivery of a therapeutic RF energy signal. The distortion is due to the electrical noise created by the delivery of the therapeutic RF energy signal. In contrast, jaw aperture measurement (700B) of FIG. 7B illustrates the measurements generated from the jaw angle sensor (199) during delivery of the same therapeutic RF energy signal of FIG. 7A without any distortion, which is avoided by applying method (500) of FIG. 5.

### III. Illustrative Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical system, comprising: (a) an end effector, including: (i) a plurality of electrodes configured to contact a tissue of a patient, and (ii) a first sensor configured to generate a first measurement during use; and (b) a processor configured to:
(i) control delivery of a therapeutic radio frequency (RF) energy signal based on an energy delivery algorithm to the plurality of electrodes, (ii) initiate a sensing window of time defined by pausing the delivery of the therapeutic RF energy signal for a duration of time whereafter the delivery of the therapeutic RF energy signal resumes, (iii) generate, during the sensing window, the first measurement from the first sensor, and (iv) transmit, during the sensing window, end effector data to the surgical system, wherein the end effector data comprises the first measurement from the first sensor.

### Example 2

The surgical system of Example 1, wherein the end effector further includes a first jaw and a second jaw pivotably coupled relative to the first jaw, and wherein the first measurement is a jaw aperture measurement based on a position of the first jaw relative to the second jaw.

### Example 3

The surgical system of any one or more of Examples 1 through 2, wherein the delivery of the therapeutic RF energy signal is one of: (a) advanced bipolar (ABP) energy delivery; (b) harmonic energy delivery; (c) monopolar energy delivery; and (d) bipolar energy delivery.

### Example 4

The surgical system of any one or more of Examples 1 through 3, wherein the duration of time is such that a user of the surgical system cannot perceive the pause in the delivery of the therapeutic RF energy signal.

### Example 5

The surgical system of any one or more of Examples 1 through 4, wherein the sensing window is initiated more than once during the delivery of the therapeutic RF energy signal.

### Example 6

The surgical system of Example 5, wherein the multiple initiations of the sensing window are periodic such that the multiple initiations occur at regular intervals of time during the delivery of the therapeutic RF energy signal.

### Example 7

The surgical system of Example 5, wherein the multiple initiations of the sensing window are sporadic such that the multiple initiations occur at irregular intervals of time during the delivery of the therapeutic RF energy signal.

### Example 8

The surgical system of any one or more of Examples 1 through 4, wherein the sensing window is initiated at a predetermined time during the delivery of the therapeutic RF energy signal based on the energy delivery algorithm.

### Example 9

The surgical system of Example 8, wherein the energy delivery algorithm is a harmonic advanced hemostasis algorithm comprising a sealing phase and a cutting phase, and the predetermined time occurs in between the sealing phase and the cutting phase.

### Example 10

The surgical system of any one or more of Examples 1 through 9, wherein a second measurement is generated from the first sensor during the sensing window.

### Example 11

The surgical system of any one or more of Examples 1 through 10, wherein: (a) the end effector further includes a second sensor configured to generate a second measurement during use, wherein the first measurement and the second measurement are different; and (b) the processor is further configured to generate, during the sensing window, the second measurement from the second sensor, wherein the end effector data further comprises the second measurement from the second sensor.

### Example 12

The surgical system of any one or more of Examples 1 through 11, wherein the end effector data is transmitted using one of: (a) a near-field communication (NFC) connection; (b) a Bluetooth® connection; and (c) a wired connection.

### Example 13

A method for obtaining end effector data from an end effector of a surgical instrument, comprising: (a) delivering a therapeutic radio frequency (RF) energy signal to a plurality of electrodes of the end effector based on an energy delivery algorithm; (b) initiating a sensing window of time defined by pausing the delivery of the therapeutic RF energy signal for a duration of time whereafter the delivery of the therapeutic RF energy signal resumes; (c) generating, during the sensing window, a first measurement from a first sensor, the end effector comprising the first sensor, the first sensor configured to generate the first measurement during use; and (d) transmitting, during the sensing window, the end effector data to a surgical system which comprises the surgical instrument, wherein the end effector data comprises the first measurement from the first sensor.

### Example 14

The method of Example 13, wherein the end effector includes a first jaw and a second jaw pivotably coupled relative to the first jaw, and the first measurement is a jaw aperture based on a position of the first jaw relative to the second jaw.

### Example 15

The method of any one or more of Examples 13 through 14, wherein the duration of time is such that a user of the surgical instrument cannot perceive the pause in the delivery of the therapeutic RF energy signal.

### Example 16

The method of any one or more of Examples 13 through 15, wherein the sensing window is initiated more than once during the delivery of the therapeutic RF energy signal.

### Example 17

The method of any one or more of Examples 13 through 15, wherein the sensing window is initiated at a predetermined time during the delivery of the therapeutic RF energy signal based on the energy delivery algorithm.

### Example 18

The method of Example 17, wherein the energy delivery algorithm is a harmonic advanced hemostasis algorithm comprising a sealing phase and a cutting phase, and the predetermined time occurs in between the sealing phase and the cutting phase.

### Example 19

The method of any one or more of Examples 13 through 18, further comprising generating, during the sensing window, a second measurement from a second sensor, the end effector further comprising the second sensor, the second sensor configured to generate the second measurement during use, wherein the first measurement and the second measurement are different, and wherein the end effector data further comprises the second measurement from the second sensor.

### Example 20

A surgical system comprising: (a) a waveform generator; (b) a surgical instrument, comprising: (i) an end effector, including: (A) a first jaw, (B) a second jaw pivotably coupled relative to the first jaw, (C) a plurality of electrodes configured to contact a tissue of a patient, and (D) at least one sensor configured to generate a first measurement during use, and (c) a processor configured to: (i) control delivery of a therapeutic radio frequency (RF) energy signal based on an energy delivery algorithm to the plurality of electrodes, wherein the therapeutic RF energy signal is generated by the waveform generator, (ii) initiate a sensing window of time defined by pausing the delivery of the therapeutic RF energy signal for a duration of time whereafter the delivery of the therapeutic RF energy signal resumes, (iii) generate, during the sensing window, the first measurement from the sensor, and (iv) transmit, during the sensing window, end effector data to the surgical system, wherein the end effector data comprises the first measurement from the sensor.

### IV. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of surgical systems and electrosurgical instruments, it should be understood that various teachings herein may be readily applied to a variety of other types of systems and devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical system, comprising:
(a) an end effector, including:
(i) a plurality of electrodes configured to contact a tissue of a patient, and
(ii) a first sensor configured to generate a first measurement during use; and
(b) a processor configured to:
(i) control delivery of a therapeutic radio frequency (RF) energy signal based on an energy delivery algorithm to the plurality of electrodes,
(ii) initiate a sensing window of time defined by pausing the delivery of the therapeutic RF energy signal for a duration of time whereafter the delivery of the therapeutic RF energy signal resumes,
(iii) generate, during the sensing window, the first measurement from the first sensor, and
(iv) transmit, during the sensing window, end effector data to the surgical system, wherein the end effector data comprises the first measurement from the first sensor.

2. The surgical system of claim 1, wherein the end effector further includes a first jaw and a second jaw pivotably coupled relative to the first jaw, and wherein the first measurement is a jaw aperture measurement based on a position of the first jaw relative to the second jaw.

3. The surgical system of claim 1, wherein the delivery of the therapeutic RF energy signal is one of:
(a) advanced bipolar (ABP) energy delivery;
(b) harmonic energy delivery;
(c) monopolar energy delivery; and
(d) bipolar energy delivery.

4. The surgical system of claim 1, wherein the duration of time is such that a user of the surgical system cannot perceive the pause in the delivery of the therapeutic RF energy signal.

5. The surgical system of claim 1, wherein the sensing window is initiated more than once during the delivery of the therapeutic RF energy signal.

6. The surgical system of claim 5, wherein the multiple initiations of the sensing window are periodic such that the multiple initiations occur at regular intervals of time during the delivery of the therapeutic RF energy signal.

7. The surgical system of claim 5, wherein the multiple initiations of the sensing window are sporadic such that the multiple initiations occur at irregular intervals of time during the delivery of the therapeutic RF energy signal.

8. The surgical system of claim 1, wherein the sensing window is initiated at a predetermined time during the delivery of the therapeutic RF energy signal based on the energy delivery algorithm.

9. The surgical system of claim 8, wherein the energy delivery algorithm is a harmonic advanced hemostasis algorithm comprising a sealing phase and a cutting phase, and the predetermined time occurs in between the sealing phase and the cutting phase.

10. The surgical system of claim 1, wherein a second measurement is generated from the first sensor during the sensing window.

11. The surgical system of claim 1, wherein:
(a) the end effector further includes a second sensor configured to generate a second measurement during use, wherein the first measurement and the second measurement are different; and
(b) the processor is further configured to generate, during the sensing window, the second measurement from the second sensor, wherein the end effector data further comprises the second measurement from the second sensor.

12. The surgical system of claim 1, wherein the end effector data is transmitted using one of:
(a) a near-field communication (NFC) connection;
(b) a Bluetooth® connection; and
(c) a wired connection.

13. A method for obtaining end effector data from an end effector of a surgical instrument, comprising:
(a) delivering a therapeutic radio frequency (RF) energy signal to a plurality of electrodes of the end effector based on an energy delivery algorithm;
(b) initiating a sensing window of time defined by pausing the delivery of the therapeutic RF energy signal for a duration of time whereafter the delivery of the therapeutic RF energy signal resumes;
(c) generating, during the sensing window, a first measurement from a first sensor, the end effector comprising the first sensor, the first sensor configured to generate the first measurement during use; and
(d) transmitting, during the sensing window, the end effector data to a surgical system which comprises the surgical instrument, wherein the end effector data comprises the first measurement from the first sensor.

14. The method of claim 13, wherein the end effector includes a first jaw and a second jaw pivotably coupled relative to the first jaw, and the first measurement is a jaw aperture based on a position of the first jaw relative to the second jaw.

15. The method of claim 13, wherein the duration of time is such that a user of the surgical instrument cannot perceive the pause in the delivery of the therapeutic RF energy signal.

16. The method of claim 13, wherein the sensing window is initiated more than once during the delivery of the therapeutic RF energy signal.

17. The method of claim 13, wherein the sensing window is initiated at a predetermined time during the delivery of the therapeutic RF energy signal based on the energy delivery algorithm.

18. The method of claim 17, wherein the energy delivery algorithm is a harmonic advanced hemostasis algorithm comprising a sealing phase and a cutting phase, and the predetermined time occurs in between the sealing phase and the cutting phase.

19. The method of claim 13, further comprising generating, during the sensing window, a second measurement from a second sensor, the end effector further comprising the second sensor, the second sensor configured to generate the second measurement during use, wherein the first measurement and the second measurement are different, and wherein the end effector data further comprises the second measurement from the second sensor.

20. A surgical system comprising:
(a) a waveform generator;
(b) a surgical instrument, comprising:
(i) an end effector, including:
(A) a first jaw,
(B) a second jaw pivotably coupled relative to the first jaw,
(C) a plurality of electrodes configured to contact a tissue of a patient, and
(D) at least one sensor configured to generate a first measurement during use, and
(c) a processor configured to:
(i) control delivery of a therapeutic radio frequency (RF) energy signal based on an energy delivery algorithm to the plurality of electrodes, wherein the therapeutic RF energy signal is generated by the waveform generator,
(ii) initiate a sensing window of time defined by pausing the delivery of the therapeutic RF energy signal for a duration of time whereafter the delivery of the therapeutic RF energy signal resumes,
(iii) generate, during the sensing window, the first measurement from the sensor, and
(iv) transmit, during the sensing window, end effector data to the surgical system, wherein the end effector data comprises the first measurement from the sensor.
